(19) United States
(12) Patent Application Publication
(10) Pub. No.: US 2024/0342503 A1
(43) Pub. Date: Oct. 17, 2024

(54) STIMULI-RESPONSIVE ANTIMICROBIAL INDWELLING MEDICAL DEVICE

(71) Applicant: Becton, Dickinson and Company, Franklin Lakes, NJ (US)

(72) Inventors: Amanda Lingle, Salt Lake City, UT (US); Megan Brooks, Salt Lake City, UT (US); Morgan Izykowski, Midvale, UT (US); David Colton Jackson, Cottonwood Heights, UT (US); Joseph Aamodt, Salt Lake City, UT (US)

(21) Appl. No.: 18/133,927

(22) Filed: Apr. 12, 2023

(51) Int. Cl. A61N 5/06; A61B 5/00; A61B 5/01; A61B 5/0538; A61B 5/145

(52) U.S. Cl. CPC A61N 5/0624; A61B 5/01; A61B 5/0538; A61B 5/14503; A61B 5/14539; A61B 5/14542; A61B 5/14546; A61B 5/6852; A61N 5/0601; A61N 2005/063; A61N 2005/0651

(57) ABSTRACT

An indwelling medical device with stimuli-responsive antimicrobial properties. The medical device includes a polymeric substrate having one or more light-responsive antimicrobial compounds. The medical device includes a light source to provide light to the polymeric substrate and a sensor to detect infection or bacterial colonization. A controller is connected to the light source and to the sensor. The controller controls operation of the sensor and receives sensing signals from the sensor. The controller activates the light source in response to infection detection and release the antimicrobial compound. The light-responsive antimicrobial compound may include a nitric oxide donor, such as S-nitroso-n-acetylpenacillamine, S-nitrosoglutathione, or N-diazeniumdiolate (NONOate). The light-responsive antimicrobial compound may include a photocatalyst metal oxide, such as ZnO, CuO, or TiO₂. The light-responsive antimicrobial compound may include a photosensitive dye, such as crystal violet, methylene blue ethyl violet, rose bengal, or malachite green.

### BACKGROUND

The present disclosure relates to an indwelling medical device which combines infection detection capability with stimuli-responsive antimicrobial release. The disclosed medical devices include a sensor that can detect infection, a stimulus-generating device that is activated when infection is detected, and a stimulus-responsive antimicrobial compound which is released upon exposure to the stimulus.

Indwelling medical devices include medical devices which are susceptible to bacterial colonization and infection due to prolonged contact with blood or body fluids. Examples of indwelling medical devices include, but are not limited to, catheters, such as central venous catheters (CVCs) and peripherally inserted central catheters (PICCs), urinary catheters, dialysis catheters, as well as auxiliary equipment and tubing that contacts blood, such as blood infusion equipment, plasma collection equipment, dialysis equipment, etc.

Catheters are life saving devices that have become a standard of care. Catheter-related bloodstream infection (CRBSI) and central line-associated bloodstream infection (CLABSI) are caused by the colonization of microorganisms in patients with intravascular catheters and access devices. These infections are an important cause of illness and excess medical costs, as approximately 250,000-400,000 cases of central venous catheter (CVC) associated bloodstream infections occur annually in US hospitals. In addition to the monetary costs, these infections are associated with anywhere from 20,000 to 100,000 deaths each year. Despite guidelines to help reduce healthcare associated infections (HAIs), catheter-related bloodstream infections continue to plague our healthcare system.

Multiple approaches are utilized to mitigate the occurrence of these infections-namely proper insertion site cleaning, good catheter placement practice, and use of antimicrobial agents in or on the catheter tubing to suppress microbial growth.

A majority of the commercially available catheters that are used today do not have any antimicrobial action. To provide antimicrobial properties, antimicrobial agents have been immobilized into the catheter matrix or coated onto the catheter surface. These catheters, however, have given less than satisfactory results.

Accordingly, there is a need in the art for catheters and other implantable medical devices having improved antimicrobial capabilities.

The subject matter claimed herein is not limited to embodiments that solve any disadvantages or that operate only in environments such as those described above. Rather, this background is only provided to illustrate one example technology area where some implementations described herein may be practiced.

### SUMMARY

The present invention has been developed in response to problems and needs in the art that have not yet been fully resolved by currently available indwelling medical devices. The disclosed medical devices combine infection detection and active means of combatting infection.

One general aspect of the disclosure includes an indwelling medical device with stimuli-responsive antimicrobial properties. The medical device may include a polymeric substrate comprising one or more stimuli-responsive antimicrobial compounds. A stimulus source provides stimulus to the one or more stimuli-responsive antimicrobial compounds of the polymeric substrate. A sensor detects infection. A controller connected to the stimulus source and to the sensor activates the stimulus source in response to infection detection. The stimulus interacts with the one or more stimuli-responsive antimicrobial compounds to cause release of the antimicrobial compound.

Implementations of the indwelling medical device may include one or more of the following features.

Non-limiting examples of sensors that can detect infection include temperature sensors, pH sensors, procalcitonin (PCT) sensors, lactate sensors, and oxygen sensors. For instance, a change in body temperature may indicate infection. A change in blood or urine pH may indicate infection. Detecting an elevated serum procalcitonin level may indicate infection. Detecting lactate may indicate infection. A change in oxygen level may indicate infection.

Non-limiting examples of sensors that can detect bacterial colonization include impedance detectors.

In some embodiments, the stimulus is light. In some embodiments, the stimuli-responsive antimicrobial compounds are light-responsive antimicrobial compounds. The light may be produced by a light source. In some embodiments, the light source is integrated into the indwelling medical device. In some embodiments, the light source is a light emitting optical fiber. The optical fiber may include a light-diffusing tip to disperse light towards the substrate containing one or more light-responsive antimicrobial compounds. The optical fiber may be configured to receive light from a laser, laser diode, LED, or other light-producing device.

In some embodiments, the light source is a LED or plurality of LEDs configured to disperse light towards the polymeric substrate which contains one or more light-responsive antimicrobial compounds. The light source may be in electrical communication with a controller which provides operational power and control of the light source.

In some embodiments, the light source is provided through an insertable instrument, such as a stylet.

Non-limiting examples of stimuli-responsive antimicrobial compounds include nitric oxide donors, photocatalyst metal oxides, and photosensitive dyes.

Non-limiting examples of nitric oxide donors include S-nitroso-n-acetyl penacillamine (SNAP), S-nitrosoglutathione (GSNO), N-diazeniumdiolate (NONOate), and mixtures thereof.

Non-limiting examples of photocatalyst metal oxides include zinc oxide (ZnO), copper oxide (CuO), and titanium dioxide (TiO₂).

Non-limiting examples of photosensitive dyes include crystal violet, methylene blue, ethyl violet, rose bengal, malachite green, and mixtures thereof.

In some embodiments, the polymeric substrate comprises a polymeric coating, wherein the polymeric coating comprises a light-responsive antimicrobial compound.

In some embodiments, the indwelling medical device is a catheter. In some embodiments, the catheter is selected from peripheral intravenous catheter (PIVC), a peripherally inserted central catheter (PICC), a midline catheter, a central venous catheter (CVC), a urinary catheter, and a dialysis catheter.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A illustrates a general representation of the operation of a disclosed indwelling medical device when no infection is detected.

FIG. 1B illustrates a general representation of the operation of a disclosed indwelling medical device when an infection is detected.

FIGS. 2A-2C illustrate embodiments of an indwelling medical device with stimuli-responsive antimicrobial properties.

FIGS. 3A-3C are cross-sectional representations of a polymeric substrate of an indwelling medical device.

FIG. 4 is a simplified illustration of an impedance detector probe system.

### DESCRIPTION OF EMBODIMENTS

The disclosure relates to an indwelling medical device which combines infection detection capability with stimuli-responsive antimicrobial release. By combining infection detection capability with stimuli-responsive antimicrobial technology, the disclosed medical devices provide all-in-one infection care feature for indwelling medical devices. When incorporated into polymeric substrates, one or more stimuli-responsive antimicrobial compounds can retain tunable, broad-spectrum antimicrobial activity against clinically relevant pathogens. A sensor detects infection or bacterial colonization and activates a stimulus generating system, which produces a stimulus. The stimulus promotes release of one or more stimuli-responsive antimicrobial compounds to provide antimicrobial activity. The disclosed “smart” stimuli-responsive system combines self-monitoring and self-combatting antimicrobial capabilities into a single platform.

In some non-limiting embodiments, the stimulus is light and the stimuli-responsive antimicrobial compounds are light-responsive antimicrobial compounds. The light may be produced by a light source. In some embodiments, the light source is integrated into the indwelling medical device. In some embodiments, the light source is a light emitting optical fiber. The optical fiber may include a light-diffusing tip to disperse light towards the substrate containing one or more light-responsive antimicrobial compounds. The optical fiber may be configured to receive light from a laser, laser diode, LED, or other light-producing device.

In some embodiments, the light source is a LED or plurality of LEDs configured to disperse light towards the substrate which contains one or more light-responsive antimicrobial compounds. The light source may be in electrical communication with a power supply via lead wires.

In some embodiments, the light source is provided through an insertable instrument, such as a stylet.

Non-limiting examples of stimuli-responsive antimicrobial compounds include nitric oxide donors, photocatalyst metal oxides, and photosensitive dyes.

Non-limiting examples of nitric oxide donors include S-nitroso-n-acetyl penacillamine (SNAP), S-nitrosoglutathione (GSNO), N-diazeniumdiolate (NONOate), and mixtures thereof.

Non-limiting examples of photocatalyst metal oxides include zinc oxide (ZnO), copper oxide (CuO), and titanium dioxide (TiO₂).

Non-limiting examples of photosensitive dyes include crystal violet, methylene blue, ethyl violet, rose bengal, malachite green, and mixtures thereof.

The disclosed invention may include two or more different stimuli-responsive antimicrobial compounds.

Non-limiting examples of sensors that can detect infection include temperature sensors, pH sensors, procalcitonin (PCT) sensors, lactate sensors, and oxygen sensors. For instance, a change in body temperature may indicate infection. A change in blood or urine pH may indicate infection. Detecting an elevated serum procalcitonin level may indicate infection. Detecting lactate may indicate infection. A change in oxygen level may indicate infection.

Non-limiting examples of sensors that can detect bacterial colonization include impedance detectors.

Table 1 summarizes non-limiting examples of infection detection and light-responsive antimicrobial technologies.

TABLE 1

| | | Stimuli-Responsive Antimicrobial | |
|---|---|---|---|
| | Stimulus-generating | Compounds | |
| Infection Detection Sensors | Systems | Agent | Stimuli |
| Temperature | Light-emitting optical | Nitric Oxide Donors (S-nitroso-n- | Light |
| pH | fiber | acetyl penacillamine, S- | |
| Impedance | LED | nitrosoglutathione, N- | |
| Hormone (lactate, | Insertable instrument | diazeniumdiolate) | |
| procalcitonin) | containing light source | Photocatalyst Metal Oxides | Light |
| Oxygen | | (ZnO, CuO, TiO₂) | |
| | | Photosensitive Dyes (crystal | Light |
| | | violet, methylene blue, ethyl | |
| | | violet, rose bengal, malachite | |
| | | green,) | |

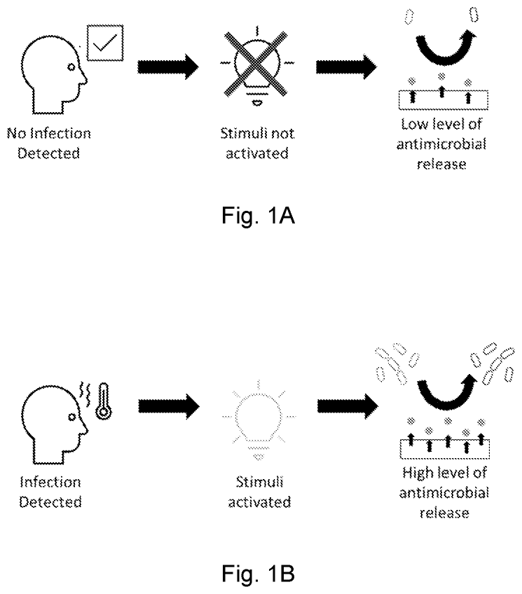

FIG. 1A illustrates the operation of a disclosed indwelling medical device when no infection is detected. The indwelling medical device includes a polymeric substrate containing one or more stimuli-responsive antimicrobial compounds. The medical device further includes a sensor to detect an infection or bacterial colonization. The sensor is connected to a stimulus-generating system, such as a light source. When no infection is detected, the stimulus-generating system is not activated. In this situation, the medical device provides passive low level of antimicrobial compound release.

FIG. 1B illustrates the operation of a disclosed indwelling medical device when an infection is detected. When an infection is detected, the stimulus-generating system is activated. In this case, the stimulus-generating system is a light source which provides light to the polymeric substrate containing the one or more stimuli-responsive antimicrobial compounds. The light stimulus promotes release of the one or more stimuli-responsive antimicrobial compounds to provide active high level antimicrobial compound release to combat invading pathogens.

The disclosed stimuli-responsive antimicrobial indwelling medical devices combine infection detection and combatting bacterial colonization into a single platform. Prior technologies preventing/combatting bacterial colonization rely on either passive, uncontrollable release of antimicrobial agents or require manual activation of different stimuli to activate the release of antimicrobial agents from the impregnated surface. Similarly, while infection detection technologies are known, they do not automatically activate means of combatting infection in the medical device.

The disclosed stimuli-responsive antimicrobial indwelling medical devices provide an implicit monitoring of bacterial adhesion directly at the medical device surface with a primary sensor to detect an infection or bacterial colonization, which activates a secondary stimuli-generating system to produce a stimulus, which produces a tertiary or reactive response in a substrate containing stimuli-responsive antimicrobial compounds to release the antimicrobial compounds at the site of activity. This allows for an early detection and passive response outside of clinical user intervention to improve device functionality and clinical outcomes. Burden and reliance on external lab testing and hospital infrastructure are reduced. Necessity of medical device removal and complications of repeated placement procedures are mitigated. Additionally, monitoring at the direct site of infection allows for potential for output of information to the clinical user to take further measures to provide better care and outcomes for their patients.

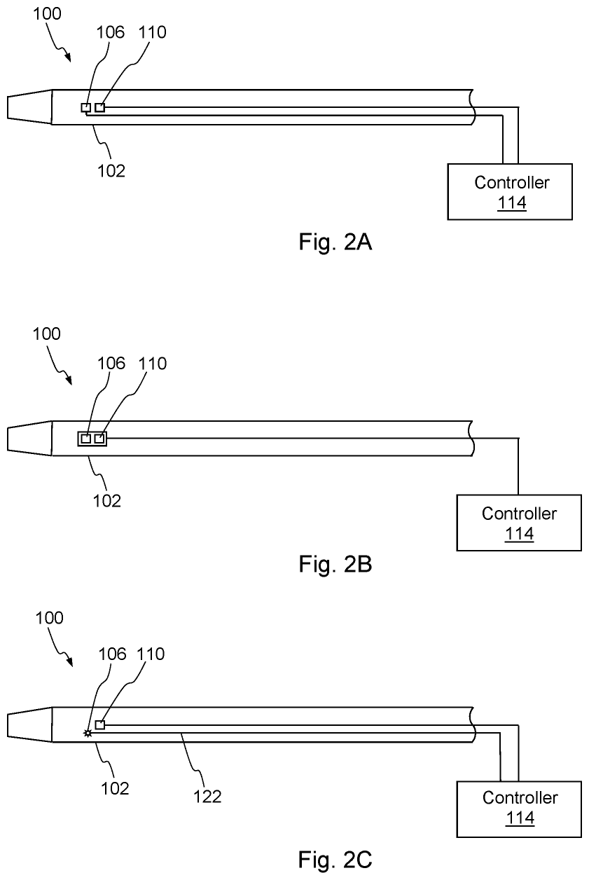

FIGS. 2A-2C illustrate an indwelling medical device 100 with stimuli-responsive antimicrobial properties. The indwelling medical device 100 shown in FIGS. 2A-2C is represented by a distal end portion of an indwelling catheter. Non-limiting examples of indwelling catheters include peripheral intravenous catheter (PIVC), a peripherally inserted central catheter (PICC), a midline catheter, a central venous catheter (CVC), a urinary catheter, and a dialysis catheter.

The indwelling medical device 100 includes a polymeric substrate 102 comprising a light-responsive antimicrobial compound. The indwelling medical device 100 includes a light source 106 to provide light to the polymeric substrate 102. The indwelling medical device 100 includes a sensor 110 to detect infection. The indwelling medical device 100 includes a controller 114 connected to the light source and to the sensor to activate the light source in response to infection detection and promote release the antimicrobial compound.

In some embodiments, the light-responsive antimicrobial compound is a nitric oxide releasing compound. Nitric oxide is an effective broad-spectrum antimicrobial and homeostasis agent for preventive and therapeutic applications. Any physiologically compatible nitric oxide releasing compound may be used herein. Non-limiting examples of nitric oxide releasing compounds include s-nitroso-n-acetylpenicillamine (SNAP), s-nitrosoglutathione (GSNO), N-diazeniumdiolate (NONOate), and mixtures thereof.

In some embodiments, the light-responsive antimicrobial compound a photocatalyst metal oxide. In some embodiments, the photocatalyst metal oxide is selected from zinc oxide (ZnO), copper oxide (CuO), titanium dioxide (TiO₂), and mixtures thereof.

In some embodiments, the light-responsive antimicrobial compound comprises a photosensitive dye. In some embodiments, the photosensitive dye is selected from crystal violet, methylene blue, ethyl violet, rose bengal, malachite green, and mixtures thereof.

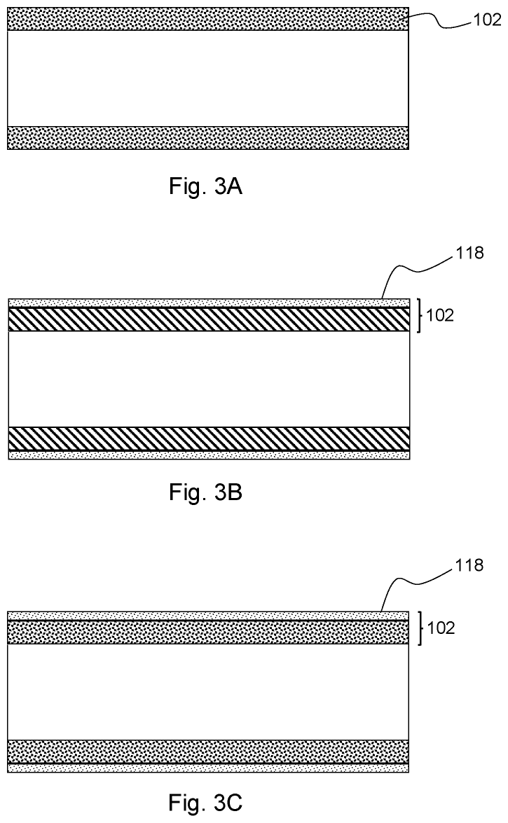

The polymeric substrate 102 may be a physiologically compatible polymeric material from which the medical device 100 is fabricated. One non-limiting example of a physiologically compatible polymeric material includes polyurethane. FIGS. 3A-3C show cross-sectional representations of a polymeric substrate 102 of an indwelling medical device in the form of a catheter.

FIG. 3A shows a cross-sectional representation of a polymeric substrate 102 containing a light-responsive antimicrobial compound. In some embodiments, the light-responsive antimicrobial compound may be incorporated into the physiologically compatible polymeric material from which the medical device is fabricated. In some embodiments, the polymeric substrate 102 may be impregnated with the light-responsive antimicrobial compound after the medical device is fabricated.

FIG. 3B shows a cross-sectional representation of a polymeric substrate 102 having a polymeric coating 118 comprising a light-responsive antimicrobial compound. The polymeric coating 118 may be applied to the polymeric substrate 102 or directly to the medical device 100.

In some embodiments, the polymeric substrate is made to contain the light-responsive antimicrobial compound by a combination of fabricating, impregnating, and/or coating with the light-responsive antimicrobial compound. FIG. 3C shows a cross-sectional representation of a polymeric substrate 102 containing a light-responsive antimicrobial compound and a polymeric coating 118 containing a light-responsive antimicrobial compound. The light-responsive antimicrobial compounds may be the same or different.

In all of the embodiments disclosed herein, more than one light-responsive antimicrobial compound may be used to provide enhanced antimicrobial activity.

The polymeric substrate 102 may be impregnating with the light-responsive antimicrobial compound by exposing the polymeric substrate 102 of the medical device to a solvent having the light-responsive antimicrobial compound dissolved therein. The medical device is exposed to the solvent solution for sufficient time to permit the light-responsive antimicrobial compound to penetrate the medical device. The impregnating step may occur at room temperature. The impregnating step may occur at a temperature in the range from about 25 to 55° C. Any solvent that is compatible with the light-responsive antimicrobial compound and medical device may be used. The solvent should not cause polymer degradation. The solvent should also be effectively removed by evaporation. Unevaporated solvent should be avoided.

The light-responsive antimicrobial compound may be dissolved in tetrahydrofuran (THF), dioxolane, methyl ethyl ketone (MEK), methanol, ethanol, isopropyl alcohol, water, or combinations thereof. The medical device may be soaked in these solutions containing the light-responsive antimicrobial compound for sufficient time to impregnate the medical device with the light-responsive antimicrobial compound. The exposure time may range between 5 minutes and 24 hours.

The medical device may be further impregnated with a catalyst to facilitate release of the light-responsive antimicrobial compound. Non-limiting examples of such catalysts include copper, iron, zinc, selenium, and silver. The catalyst may be impregnated into the medical device by exposing the medical device to a solvent having the catalyst dissolved therein. The catalyst may be impregnated into the medical device using the same solvent system as the light-responsive antimicrobial compound, discussed above, either during the same impregnation step, a subsequent impregnation step, or a prior impregnation step. The medical device is exposed to the solvent solution for sufficient time to permit the catalyst to penetrate the medical device. The impregnating step may occur at room temperature. The impregnating step may occur at a temperature in the range from about 25 to 55° C. Any solvent that is compatible with the catalyst and medical device may be used, including those described above in relation to the light-responsive antimicrobial compound.

The medical device may be further impregnated with one or more additional light-responsive antimicrobial compounds.

In one embodiment, the polymeric substrate 102 may comprise a polymeric coating applied to the medical device containing a light-responsive antimicrobial compound. The coating may be achieved by dip-coating. The coating step may be accomplished by dip coating the medical device in a polymer solution, such as polyurethane, and a light-responsive antimicrobial compound in a suitable solvent. The light-responsive antimicrobial compound may have a concentration in the solvent from 1 to 20 wt./vol. %. Any solvent that is compatible with the light-responsive antimicrobial compound, the polymer, and medical device may be used. The solvent should not cause polymer degradation. The solvent should also be effectively removed by evaporation. Unevaporated solvent should be avoided. The solvent for the dip coating polymer solution may comprise methanol, dioxolane, and mixtures thereof. In a non-limiting embodiment, the solvent comprises from 10 vol. % to 25 vol. %, methanol and from 75 vol. % to 90 vol. % dioxolane. The coating step may occur at room temperature. The coating step may occur at a temperature in the range from about 25 to 50° C.

The indwelling medical device 100 includes a light source 106 to provide light to the polymeric substrate 102 which contains one or more light-responsive antimicrobial compounds. The light source 106 is connected to the controller 114 to control operation of the light source 106.

FIGS. 2A and 2B show an electrically powered light source 106, such as a LED or plurality of LEDs, configured to disperse light towards the polymeric substrate 102. The controller 114 may comprise a suitable power supply in electrical communication with the light source. The LED emission spectrum may be tuned to promote stimulation and release of the one or more light-responsive antimicrobial compounds present in the polymeric substrate 102.

FIG. 2B shows an embodiment where the light source 106 and sensor 110 are integrated into a single electronic device coupled to the controller 114.

In some embodiments, the light source is a light emitting optical fiber 122, as shown in FIG. 2C. The optical fiber may include a light-diffusing tip to disperse light towards the polymeric substrate 102. The controller 114 may comprise a suitable light-producing device, such as a laser, laser diode, or LED, to introduce light into the optical fiber 122. The emission spectrum of the light-producing device may be tuned to promote stimulation and release of the one or more light-responsive antimicrobial compounds present in the polymeric substrate 102.

In some embodiments, such as shown in FIGS. 2A-2C, the light source 106 is integrated into the indwelling medical device 100. In some embodiments, the light source 106 is coupled to an insertable instrument, such as a stylet. The light source emission spectrum may be tuned to promote stimulation and release of the one or more light-responsive antimicrobial compounds present in the polymeric substrate 102.

The indwelling medical device 100 includes a sensor 110 to detect infection. The sensor 110 is connected to the controller 114 to control operation of the sensor and to receive sensing signals from the sensor 110. If the sensor 110 detects infection, then the controller activates the light source to promote release of the light-responsive antimicrobial compound.

Different types of sensors may be used to detect infection. The sensor 110 may comprises a temperature sensor to detect a temperature change, which indicates infection. The sensor 110 may comprises a pH sensor to detect a pH change, which indicates infection. The sensor 110 may comprises a procalcitonin (PCT) sensor, which indicates infection. The sensor 110 may comprises a lactate sensor, which indicates infection. The sensor 110 may comprises an oxygen sensor to detect an oxygen level, which indicates infection.

Another type of sensor 110 which may be used herein is an impedance detector to detect a change in impedance of fluid in or around the medical device 100. A change of impedance can indicate bacterial adhesion or bacterial colonization. Impedance is the combined effect of ohmic resistance and reactance of the media and space in a system.

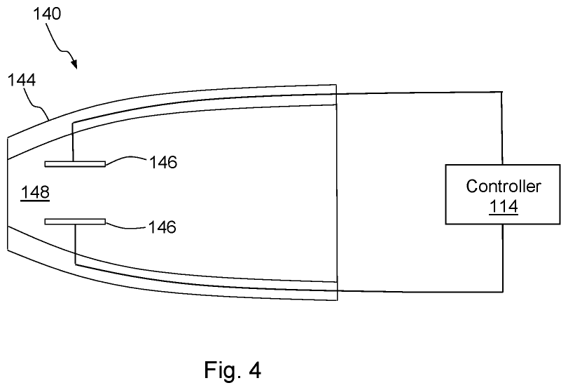

With advances in manufacturing and smaller electronics, an impedance detector may be molded directly into a catheter tip. FIG. 4 is a simplified illustration showing an impedance detector probe system 140 disposed at a catheter tip 144. The impedance detector can be molded into the extrusion walls of the catheter with simple electrode probes 146 crossing over and accessing the luminal space 148. The electrode probes 146 are electrically connected to controller 114. The controller 114 provides a small measurable current and voltage to the electrode probes 146 to enable impedance measurement. A change in impedance is measured by the controller 114. Changes in impedance at the catheter surface and surrounding media within the luminal space 148 may indicate bacterial colonization.

Locked solutions within the catheter luminal space 148 can provide for a suitable ionic media between the probes. As foreign bodies, including bacteria, ingress into the luminal space 148 and walls, the ohmic resistance across the media system and catheter surface changes across the electrode probes 146. Bacterial adhesion will directly interfere with the media space between the electrode probes 146 and cause an increase in measured impedance.

The indwelling medical device 100 includes a controller 114 connected to the light source and to the sensor to activate the light source in response to infection detection and release the antimicrobial compound. The controller 114 may include memory to record the status and operation of the sensor 110 and light source 106. The controller 114 may include a visual indicator of the status and operation of sensor 110 and light source 106.

### Embodiments

Various embodiments are listed below. It will be understood that the embodiments listed below may be combined with all aspects and other embodiments in accordance with the scope of the invention.

Embodiment 1. An indwelling medical device with stimuli-responsive antimicrobial properties comprising: a polymeric substrate comprising a light-responsive antimicrobial compound; a light source to provide light to the polymeric substrate; a sensor to detect infection or bacterial colonization; and a controller connected to the light source and to the sensor to activate the light source in response to infection detection and release the antimicrobial compound.

Embodiment 2. The indwelling medical device of Embodiment 1, wherein the sensor comprises a temperature sensor to detect a temperature change.

Embodiment 3. The indwelling medical device of Embodiments 1, wherein the sensor comprises a pH sensor to detect a pH change.

Embodiment 4. The indwelling medical device of Embodiment 1, wherein the sensor comprises an impedance detector to detect bacterial colonization.

Embodiment 5. The indwelling medical device of Embodiment 1, wherein the sensor comprises a procalcitonin (PCT) sensor.

Embodiment 6. The indwelling medical device of Embodiment 1, wherein the sensor comprises a lactate sensor.

Embodiment 7. The indwelling medical device of Embodiment 1, wherein the sensor comprises an oxygen sensor.

Embodiment 8. The indwelling medical device according to any of Embodiments 1-7, wherein the light source comprises an optical fiber connected to the light source and which directs light to the polymeric substrate.

Embodiment 9. The indwelling medical device according to any of Embodiments 1-7, wherein the light source is a LED integrated into the indwelling medical device.

Embodiment 10. The indwelling medical device according to any of Embodiments 1-7, wherein the light source is coupled to an insertable instrument.

Embodiment 11. The indwelling medical device of any preceding Embodiment, wherein the light-responsive antimicrobial compound comprises a nitric oxide donor.

Embodiment 12. The indwelling medical device of Embodiment 11, wherein the nitric oxide donor is selected from S-nitroso-n-acetylpenacillamine, S-nitrosoglutathione, N-diazeniumdiolate (NONOate), and mixtures thereof.

Embodiment 13. The indwelling medical device according to any of Embodiments 1-10, wherein the antimicrobial compound comprises a photocatalyst metal oxide.

Embodiment 14. The indwelling medical device of Embodiment 13, wherein the photocatalyst metal oxide is selected from zinc oxide (ZnO), copper oxide (CuO), titanium dioxide (TiO₂), and mixtures thereof.

Embodiment 15. The indwelling medical device according to any of Embodiments 1-10, wherein the antimicrobial compound comprises a photosensitive dye.

Embodiment 16. The indwelling medical device of Embodiment 15, wherein the photosensitive dye is selected from crystal violet, methylene blue, ethyl violet, rose bengal, malachite green, and mixtures thereof.

Embodiment 17. The indwelling medical device of any preceding Embodiment, wherein the polymeric substrate comprises a polymeric coating, wherein the polymeric coating comprises a light-responsive antimicrobial compound.

Embodiment 18. The indwelling medical device of any preceding Embodiment, wherein the indwelling medical device is a catheter.

Embodiment 19. The indwelling medical device of Embodiment 18, wherein the catheter is selected from a peripheral intravenous catheter (PIVC), a peripherally inserted central catheter (PICC), a midline catheter, a central venous catheter (CVC), a urinary catheter, and a dialysis catheter.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. An indwelling medical device with stimuli-responsive antimicrobial properties comprising:
a polymeric substrate comprising a light-responsive antimicrobial compound;
a light source to provide light to the polymeric substrate;
a sensor to detect infection; and
a controller connected to the light source and to the sensor to activate the light source in response to infection detection and release the light-responsive antimicrobial compound.

2. The indwelling medical device of claim 1, wherein the sensor comprises a temperature sensor to detect a temperature change.

3. The indwelling medical device of claim 1, wherein the sensor comprises a pH sensor to detect a pH change.

4. The indwelling medical device of claim 1, wherein the sensor comprises an impedance detector to detect bacterial colonization.

5. The indwelling medical device of claim 1, wherein the sensor comprises a procalcitonin (PCT) sensor.

6. The indwelling medical device of claim 1, wherein the sensor comprises a lactate sensor.

7. The indwelling medical device of claim 1, wherein the sensor comprises an oxygen sensor.

8. The indwelling medical device of claim 1, wherein the light source comprises an optical fiber connected to the light source and which directs light to the polymeric substrate.

9. The indwelling medical device of claim 1, wherein the light source is a LED integrated into the indwelling medical device.

10. The indwelling medical device of claim 1, wherein the light source is coupled to an insertable instrument.

11. The indwelling medical device of claim 1, wherein the light-responsive antimicrobial compound comprises a nitric oxide donor.

12. The indwelling medical device of claim 11, wherein the nitric oxide donor is selected from S-nitroso-n-acetylpenacillamine, S-nitrosoglutathione, N-diazeniumdiolate (NONOate), and mixtures thereof.

13. The indwelling medical device of claim 1, wherein the light-responsive antimicrobial compound comprises a photocatalyst metal oxide.

14. The indwelling medical device of claim 13, wherein the photocatalyst metal oxide is selected from zinc oxide (ZnO), copper oxide (CuO), titanium dioxide (TiO₂), and mixtures thereof.

15. The indwelling medical device of claim 1, wherein the light-responsive antimicrobial compound comprises a photosensitive dye.

16. The indwelling medical device of claim 15, wherein the photosensitive dye is selected from crystal violet, methylene blue, ethyl violet, rose bengal, malachite green, and mixtures thereof.

17. The indwelling medical device of claim 1, wherein the polymeric substrate comprises a polymeric coating, wherein the polymeric coating comprises a light-responsive antimicrobial compound.

18. The indwelling medical device of claim 1, wherein the indwelling medical device is a catheter.

19. The indwelling medical device of claim 18, wherein the catheter is selected from a peripheral intravenous catheter (PIVC), a peripherally inserted central catheter (PICC), a midline catheter, a central venous catheter (CVC), a urinary catheter, and a dialysis catheter.
